(19) United States
(12) Patent Application Publication
(10) Pub. No.: US 2025/0031957 A1
(43) Pub. Date: Jan. 30, 2025

(54) FLUID TRACKING IN WET AMD PATIENTS USING THICKNESS CHANGE ANALYSIS

(71) Applicants: Carl Zeiss Meditec, Inc., Dublin, CA (US); Carl Zeiss Meditec AG, Jena (DE)

(72) Inventors: Mahsa Darvishzadeh Varcheie, Fremont, CA (US); Simon Bello, Pleasant Hill, CA (US)

(73) Assignees: Carl Zeiss Meditec, Inc., Dublin, CA (US); Carl Zeiss Meditec AG, Jena, TH (DE)

(21) Appl. No.: 18/716,626

(22) PCT Filed: Dec. 23, 2022
(86) PCT No.: PCT/EP2022/087708

§ 371 Date: Jun. 5, 2024

Related U.S. Application Data
(60) Provisional application No. 63/294,000, filed on Dec. 27, 2021.

(51) Int. Cl. A61B 3/10; A61B 3/12

(52) U.S. Cl. CPC A61B 3/102; A61B 3/1225

(57) ABSTRACT

The present System/Method/Device uses a (e.g., self-use) OCT system for personalized monitoring of wet AMD patients. A patient self-administers an OCT scan, which is then automatically analyzed and notifies the patient and/or a designated authorized doctor/technician if there is a need for application of medication or professional medical attention.

### FIELD OF INVENTION

The present invention is generally directed to a system and method for (e.g., remote) monitoring a wet AMD patient and determining when a level of fluid in the eye requires a doctor visit and/or medical treatment.

### BACKGROUND

OCT is a non-invasive imaging technique that uses light waves to penetrate into tissue and produce image information at different depths within the tissue, such as an eye. Generally, an OCT system is an interferometric imaging system based on detecting the interference of a reference beam and backscattered light from a sample illuminated by an OCT beam. Each scattering profile in the depth direction (e.g., z-axis or axial direction) may be reconstructed individually into an axial scan, or A-scan. Cross-sectional slice images (e.g., two-dimensional (2D) bifurcating scans, or B-scans) and volume images (e.g., 3D cube scans, or C-scans) may be built up from multiple A-scans acquired as the OCT beam is scanned/moved through a set of transverse (e.g., x-axis and/or y-axis) locations on the sample. When applied to the retina of an eye, OCT generally provides structural data that, for example, permits one to view, at least in part, distinctive tissue layers and vascular structures of the retina. OCT angiography (OCTA) expands the functionality of an OCT system to also identify (e.g., render in image format) the presence, or lack, of blood flow in retinal tissue. For example, OCTA may identify blood flow by identifying differences over time (e.g., contrast differences) in multiple OCT scans of the same retinal region, and designating differences in the scans that meet predefined criteria as blood flow.

An OCT system also permits construction of a planar (2D), frontal view (e.g., en face) image of a select portion of a tissue volume (e.g., a target tissue slab (sub-volume) or target tissue layer(s), such as the retina of an eye). Examples of other 2D representations (e.g., 2D maps) of ophthalmic data provided by an OCT system may include layer thickness maps and retinal curvature maps. For example, to generate layer thickness maps, an OCT system may use en face images, 2D vasculature maps of the retina, and multilayer segmentation data. Thickness maps may be based, at least in part, on measured thickness difference between retinal layer boundaries. Vasculature maps and OCT en face images may be generated, for example, by projecting on to a 2D surface a sub-volume (e.g., tissue slab) defined between two layer-boundaries. The projection may use the sub-volume's mean, sum, percentile, or other data aggregation method. Thus, the creation of these 2D representations of a 3D volume (or sub-volume) data often relies on the effectiveness of automated segmentation algorithms to identify the layers upon which the 2D representations are based.

Wet macular degeneration (or wet age-related macular degeneration, wet AMD) is a chronic condition characterized by abnormal blood vessels that grow underneath the retina. Fluid leakage of these blood vessels on back of an eye may lead to swelling and damage of the macula. If this fluid is not controlled, central vision will gradually worsen. A current treatment that helps control this fluid, and may slow the progression of wet macular degeneration, is periodic injection of anti-vascular endothelial growth factor (anti-VEGF) medication (e.g., every 4-6 weeks, depending on progress of fluid leakage). The response to anti-VEGF therapy has been found to be dependent on a variety of factors including a patient's age, lesion characteristics, lesion duration, baseline visual acuity and presence of particular genotype risk alleles. Therefore, different patients may respond differently to anti-VEGF medication, and the period between injections required by an individual patient may need to be shortened or lengthened. Typically, the only way to determine if the period between injections needs to be modified is through regular office visits to an ophthalmologist for an OCT scan, and where a physician reviews a patient's visual acuity, OCT B-scans, and macular thickness maps to determine if an injection should be prescribed (e.g., warranted). Use of professional-level OCT systems (e.g., as used in doctor's offices) in AMD monitoring and a discussion of how retinal thickness variation may affect visual acuity may be found in U.S. Pat. No. 7,301,644 and in “Associations of Variation in Retinal Thickness with Visual Acuity and Anatomic Outcomes in Eyes with Neovascular Age-related Macular Degeneration Lesions Treated with Anti-Vascular Endothelial Growth Factor Agents,” by Evans et al., JAMA Ophthalmology, 138 (10), 1043-1051, 2020.

However, frequent doctor visits to monitor wet AMD are typically not practical. An option for remote (e.g., home) monitoring of wet AMD is desirable.

It is an object of the present invention to provide a system and method that provides personalized analysis for monitoring wet AMD that is customized to individual patients.

It is another object of the present invention to provide a system and method for remote monitoring of wet AMD.

It is a further object of the present invention to provide a system and method that automatically customizes its analysis of wet AMD to a specific patient's ophthalmic tissue characteristics.

It is still another object of the present invention to provide a system and method that assists a doctor in determining when a change in macular thickness is significant and requires treatment with medication and/or modification to a current treatment.

### SUMMARY OF INVENTION

The above objects are met in a method/system providing monitoring of a medical condition in an eye (such as age-related macular degeneration, AMD), such as by automated analysis of ophthalmic OCT scans of patients (e.g., AMD patients). The present system and method is herein presented as applied to a remote (e.g., home-use or self-applied) OCT system, which is typically a lower cost device, but may optionally be used in professional-grade OCT systems, such as used in doctor's offices or clinics to help distinguish between normal retinal thickness variations that do not require treatment (such as with anti-VEFG injection) and retinal thickness variations that are significant and require treatment.

In the present system/method for monitoring age-related macular degeneration (AMD), e.g., wet AMD, a patient's eye is scanned with an OCT system. For example, the patient may self-administer the OCT scan using a home-use (e.g., remote-care or telemedicine) OCT system. The present system automatically identifies the macula of the eye, and identifies at least one macular region of interest (ROI) (or areas/sectors of interests) within the OCT scan. For example, the present system may identify at least one sector and preferably two or more (e.g., three concentric) sectors. Optionally, the three concentric macular ROIs may roughly follow composite-grid shapes in a typical Early Treatment Diabetic Retinopathy Study (ETDRS) grid. For instance, a first macular ROI may be a “Central” area/sector that may correspond to the central subfield of an ETDRS grid and have circular/disc shape with a 1 mm diameter. A second macular ROI encircling the first macular ROI may be an “Inner” area/sector that may corresponds to a combination of all inner subfields of the ETDRS grid and have an annular shape with an inner diameter of 1 mm and an outer diameter of 3 mm. A third macular ROI encircling the second macular ROI may be an “Outer” area/sector that corresponds to a combination of all outer subfields of a typical ETDRS grid have an inner diameter of 3 mm and outer diameter of 6 mm.

The present system/method then determines a representative macular thickness measure for each of the (e.g., three) macular ROIs, where each of the representative macular thickness measure is characteristic of the overall thickness of its respective macular ROI. For example, a representative macular thickness measure may be the average macular thickness within a respective macular ROI, the highest macular thickness within a macular ROI, or the average of a predefined number (e.g., 2 or more, or the top 25% to 50%) of the highest macular thickness measures within a respective macular ROI.

The present system establishes/determines a separate lowest, previous (or patient-personalized) baseline thickness (e.g., of macula retinal tissue) for each distinct macular ROI (e.g., for the central, inner, and outer sectors). Thus, the respective patient-personalized baseline thicknesses of each distinct macular ROI may be different from each other. These patient-personalized baseline thicknesses may be based on the personal medical history of the patient whose eye is OCT scanned. For example, the patient-personalized baseline thickness of each distinct macular ROI may be based on an average of (e.g., representative) retinal macular thickness measures for each corresponding macular ROI during a dry AMD period of the eye (e.g., doctor visits wherein no injection treatment was administered). Alternatively, the patient-personalized baseline thickness of each distinct macular ROI may be based on an average of a predefined number (e.g., three) of lowest, previous (e.g., representative) retinal macular thickness measures of each respective of macular ROI. These previous retinal macular thickness measures may have been taken at previous doctor visits or taken by the same self-use OCT system used to take the current OCT scan. Optionally, the number of lowest, previous retinal macular thickness measures are measures from consecutive OCT scans at time intervals corresponding to previously scheduled macular thickness check-up times (e.g., every 4 to 6 weeks). For example, the patient-personalized baseline thickness may be based on an average of a predefined number of lowest, previous representative macular thickness measures of the macular ROI determined according to caregiver-scheduled, macular thickness check-up times, e.g., where the caregiver schedules the check-up times. As another example, the number of lowest, previous retinal macular thickness measures may be selected from (consecutive or non-consecutive) previous doctor visits where no injection of medication was administered to the eye. Further optionally, the patient-personalized baseline thickness of each macular ROI may be user-adjustable, either by freely entering any value or in intervals of fixed size within a predefined range (e.g., a range of 15 μm to 30 μm in increments of 5 μm).

The determined representative macular thickness measure for each of the macular ROIs is then compared to its corresponding patient-specific baseline thickness with a corresponding upper threshold offset and lower threshold offset. For example, an upper specification limit (USL) for a respective macular ROI may be defined as its corresponding patient-specific baseline thickness plus its corresponding upper threshold offset. Similarly, a lower specification limit (LSL) for a respective macular ROI may be defined as its corresponding patient-specific baseline thickness less its corresponding lower threshold offset. In some applications, the upper threshold offset and lower threshold offset may be individually set, or be the same for all macular ROIs. Optionally, at least one of the upper threshold offset and lower threshold offset may be based on a statistical analysis of a population of macular thickness measures from corresponding macular ROIs in a population of test eyes not including the eye of the patient, and taken during stable periods, wherein a stable period may be defined as a period of dry AMD, or a period where a test eye's macular thickness did not vary by more than a predefined percentile (e.g., 5%), or a period where no medication (e.g., an anti-VEGF injection) was applied. The statistical analysis may provide a statistical deviation (e.g., standard deviation) among the population of macular thickness measures, and if so, at least one of the upper threshold offset and lower threshold offset may be based on the statistical analysis.

In response to the representative macular thickness measure of any of the macular ROIs being higher than (or equal or not less than) its respective upper specification limit, the present system issues a signal (e.g., electronic, audio, visual, haptic, etc.) indicating an irregularity. This irregularity may be interpreted as indicating that medical attention may be advisable. For example a medical practitioner, e.g., retinal specialist or doctor, may examine the patient and provide a medical diagnoses based on a full medical examination, which may include collecting and examining OCT images and other medical data. For example, the issued signal may be an electronic message sent remotely to the patient's doctor (or doctor's office) by a telecommunication network (e.g., text message, electronic mail, internet, telephone, etc.).

In response to the representative macular thickness measure of any of the macular ROIs being lower than (or equal to) its respective lower specification limit, the patient-personalized baseline thickness of the affected macular ROI may be adjusted. For instance, it may be adjusted based on the affected macular ROI's representative macular thickness measure. In some applications, the affected patient-personalized baseline thickness is not adjusted on the first time it is lower than the lower specification limit, but rather is adjusted on the second or third consecutive time that the representative macular thickness measure of the eye is lower than the lower specification limit. The affected patient-personalized baseline thickness may be adjusted to an average of two or more representative macular thickness measures (optionally including the current value) of the eye that are also lower than the lower specification limit.

Optionally, at least one of the upper specification limit and lower specification limit of the affected macular ROI may be adjusted in response to its representative macular thickness measure being higher than (or equal to) the upper specification limit or being lower than (or equal to) the lower specification limit. For example, the upper specification limit and/or lower specification limit may be automatically adjusted (e.g., optionally by adjusting their respective offset value) by a preset incremental amount within a predefined range. Alternatively, the upper specification limit and/or lower specification limit (or their respective offset) may be automatically adjusted by incorporating the representative macular thickness measure of the affected macular ROI into a recalculation of the upper specification limit and/or lower specification limit. This recalculation may include, for example, averaging a deviation measure of the representative macular thickness measure of the affected macular ROI with the deviation measure upon which the upper specification limit and/or lower specification limit are based. Alternatively, this value may be incorporated by recalculating the statistical deviation(s) upon which the upper/lower limits are based and incorporating the representative macular thickness measure of the affected macular ROI into this recalculation. Another option is for the upper specification limit and/or lower specification limit (or their respective offset value) to be adjusted remotely by an authorized user in response to receiving an electronic message over a telecommunication network.

Other objects and attainments together with a fuller understanding of the invention will become apparent and appreciated by referring to the following description and claims taken in conjunction with the accompanying drawings.

Several publications may be cited or referred to herein to facilitate the understanding of the present invention. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIG. 1A provides an example of three designated sectors on a retina (e.g., three macular regions of interest, ROIs) in which the present invention investigates/monitors retinal thickness change, such as for determining a person's patient-specific (or patient-personalized) baseline thickness and macular thickness variation threshold levels (e.g., upper specification limit and/or lower specification limit).

FIG. 1B provides an example of a typical Early Treatment Diabetic Retinopathy Study, ETDRS, grid.

FIG. 2 shows three data plots of macular thickness (in μm) of each of the three corresponding retinal sectors (e.g., macular ROIs, as shown in FIG. 1) for all available doctor office visits of one of twenty-two wet AMD patients used in a statistical study.

FIGS. 3A and 3B show two additional data plot examples of two patients (e.g., Patient-A and Patient-B), showing macular thickness data for the central, inner, and outer sectors (macular ROIs) of FIG. 1.

FIG. 4A illustrates the standard deviation of retinal thickness on each of the central sector, inner sector, and outer sector during stable period for wet AMD (wAMD) patients.

FIG. 4B shows a table that provide a summary of normal retinal thickness variations and standard deviation over 30 stable periods of the 22 wet AMD patients.

FIGS. 5A and 5B provide an overview of injections and doctor office visits of two wAMD patients over a two-year period (FIG. 5A) and a three-years (FIG. 5B).

FIGS. 6 and 7 illustrate two stages of an exemplary workflow (e.g., to set a lower/upper specification limit) for remote monitoring of fluid leakage in wAMD patients in accord with the present invention.

FIGS. 8A, 8B, 8C, and 8D show a first example of the application of the present invention to retinal thickness data of another wAMD patient (Patient-C) over an eight-year period.

FIGS. 9A, 9B, 9C, and 9D show a second example of the application of the present invention to retinal thickness data of another wAMD patient (Patient-D) over an eight-year period.

FIG. 10 illustrates a generalized frequency domain optical coherence tomography system used to collect 3D image data of an eye suitable for use with the present invention.

FIG. 11 shows an exemplary OCT B-scan image of a normal retina of a human eye, and illustratively identifies various canonical retinal layers and boundaries.

FIG. 12 shows an example of an en face vasculature image.

FIG. 13 shows an exemplary B-scan of a vasculature (OCTA) image.

FIG. 14 illustrates an example computer system (or computing device or computer) suitable for use with the present invention.

### DESCRIPTION OF THE PREFERRED EMBODIMENTS

The ability to monitor the progress of fluid leakage in the retina is crucial for remote monitoring of wet age-related macular degeneration (wet AMD, wAMD). Remote monitoring would require a patient to regularly acquire OCT scans without need for a doctor's office visit, or an operator/technician to acquire the OCT scan or a physician (or specialized technician) to review every acquired scan. Therefore, it is desirable that a home-use or self-use or portable or remote/telemedicine OCT device/system (e.g., an OCT device used for remote monitoring) be able to monitor the progress of fluid leakage, itself. Understanding normal fluctuations of retinal physiology changes permits the present system to determine when a change is significant (e.g., irregular, or indicative of a possible need for an anti-VEGF medication and/or doctor's visit) and notify the patient's physician (e.g., locally or remotely via an electronic message/signal on an electronic display or speaker or over the Internet or other wired/wireless telecommunication network/system and/or computer network) for further medical assistance. For example a medical practitioner, e.g., retinal specialist or doctor, may examine the patient and provide a medical diagnoses based on a full medical examination, which may include collecting and examining OCT images and other medical data.

Generating macular thickness maps based on OCT scans is a technology that is implemented in many commercial OCT systems. As stated above, currently a retina specialist may use visual acuity data, B-scans, and thickness maps to drive a decision on the best treatment scenario for a patient, see for example, Amoaku et al., “Defining Response to Anti-VEGF Therapies in Neovascular AMD”, Eye 29, 721-731, 2015, herein incorporated in its entirety by reference. Moreover, the CIRRUSR and other commercial OCT systems currently perform macular thickness change analysis between any two visits that are available in their database, which helps to identify changes in each region of an Early Treatment of Diabetic Retinopathy Study (ETDRS) macular grid. However, these data are compiled from doctor visits, and thus only become available once physician acquires an OCT scan of a patient during an office visit.

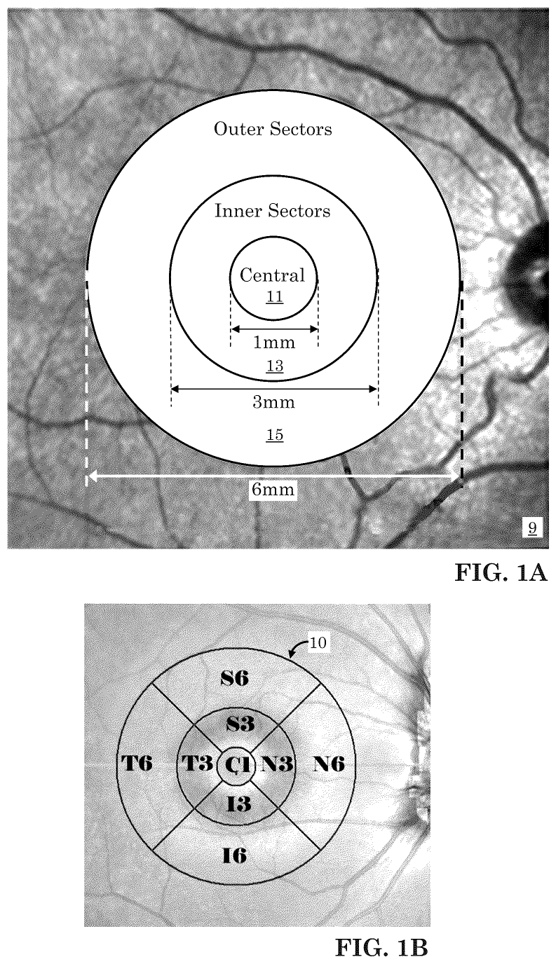

Herein, analysis of retinal thickness change over time is demonstrated as an effective tool for assessing fluid tracking in wet AMD patients. Applicants have found that by monitoring macular thickness changes over time in specific areas, the present system can determine a person's patient-personalized or patient-specific (e.g., individualized or personalized) baseline thickness (e.g., normative levels not requiring medical intervention) and patient-specific (upper/lower) threshold (e.g., upper/lower specification limit) indicative of when medical intervention is needed. FIG. 1A provides an example of three sectors (or macular areas/regions of interest, ROIs) on a retina 9 to investigate retinal thickness change, such as for determining a person's patient-specific baseline and (e.g., patient-specific) macular thickness variation threshold levels (e.g., upper specification limit and/or lower specification limit). For illustration purposes FIG. 1B provides an example of a typical ETDRS grid 10, as known in the art. In FIG. 1A, the three sectors (e.g., three concentric macular ROIs) are identified as: a Central Area 11, which may optionally correspond to the central subfield of a typical ETDRS grid (C1 in FIG. 1B), and is illustrated as a circle (or solid disc region) 11 with a diameter of 1 mm; an Inner Sector 13, which may optionally correspond to a combination of all inner subfields of an ETDRS grid (S3, N3, I3, and T3), and is illustrated as a first annulus (or shell or disc with a hole) 13 concentric with the Central Area 11 and having an inner diameter of 1 mm and outer diameter of 3 mm; and an Outer Sector 15, which may correspond to a combination of all outer subfields of an ETDRS grid (S6, N6, I6, and T6), and is here illustrated as a second annulus (or shell) concentric with the Inner Sector 13 and having an inner diameter of 3 mm and outer diameter of 6 mm. These three sectors, or regions of interest, have been found to simplify the collecting of information, but still provide enough information about fluid location (and change) for the purposes of the present invention.

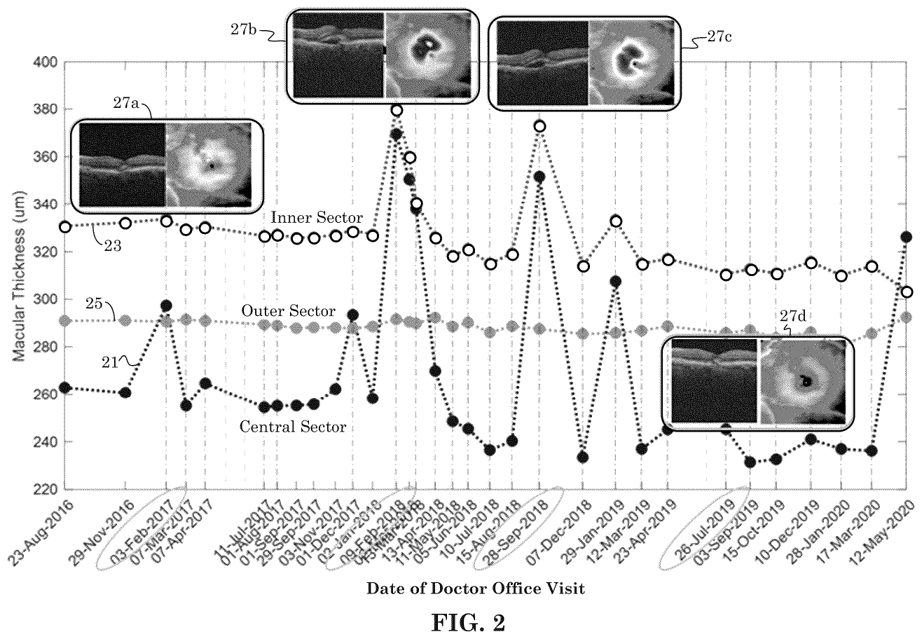

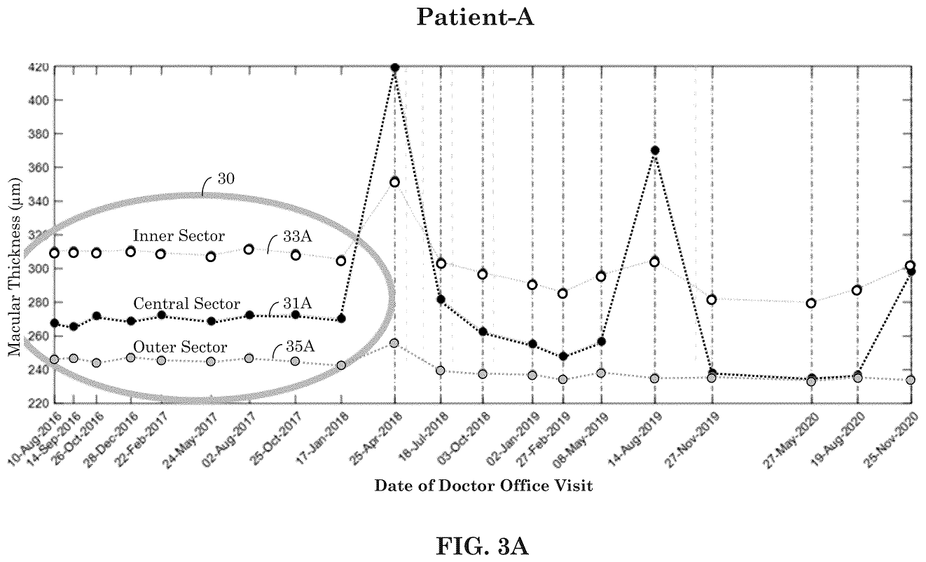

In an exemplary implementation, OCT scans of 22 patients with wet AMD collected with a Cirrus™ OCT system over the course of 3 years were reviewed. This 3-year period covered about a 6-month period of dry AMD and about a 30-month period of wet AMD (wAMD). The data was properly annotated to indicate office visits with, and without, injections (e.g., anti-VEGF injections). Macular thickness change over time was plotted for each of these 22 patients. As an example, FIG. 2 shows three plots 21, 23, and 25 of macular thickness (in μm) of each of the three corresponding sectors 11, 13, and 15 (shown in FIG. 1A) for all available office visits of one of the twenty-two wet AMD patients. For example, plot 21 follows macular thickness for the Central Area 11, plot 23 indicates the macular thickness for the Inner Sector 13, and plot 25 shows the macular thickness for the Outer Sector 15. Each of plotted macular thickness measure is a representative macular thickness measure that is characteristic of the overall thickness of its respective macular ROI. For example, multiple individual thickness measures may be determined distributed across each macular ROI, and a macular ROI's representative macular thickness measure may be based on these multiple individual macular thickness measures. For example, a macular ROI's representative thickness measure may be determined as the average of all its individual macular thickness measures, or its highest individual macular thickness measure, or the average of 2 or more (e.g., 10% to 50%) of its highest-most individual macular thickness measures, or a combination of these approaches.

In FIG. 2, every dot on the three plots 21, 23, and 25 indicates a representative retinal (e.g., macular) thickness measure, as determined at an office visit when an OCT scan was taken. To better understand macular thickness changes, pairs of corresponding central B-scans and macular thickness maps 27a, 27b, 27c, and 27d are shown for four select office visits, as indicated by encircled dates on the horizontal axis of the plot. Vertical dash lines indicate office visits in which an injection was administered, and the absence of a vertical dashed line at any office visit indicates that no injection was given. In this example, for the first 6 months (from August of 2016 to February of 2017) this particular patient was in a dry period (e.g., dry AMD, where no fluid buildup was observed), but on Feb. 3, 2017 the patient received a first injection, indicating that the patient's dry AMD had converted/progressed to wet AMD.

As used herein, the term “stable period” is defined as a period that covers multiple (or alternatively, three or more) office visits where retinal thickness at none of the sectors has significantly changed (e.g., change not greater than 5%, or no injection of medication was prescribed for, or administered to, the eye), and the patient most probably did not require an injection. Generally during a stable period, there is no significant fluid pocket visible on B-scans, as well. It has been observed that if a patient responds well to medication, this will become evident (e.g., by entering a stable period) typically about one month after a patient has received three consecutive injections. It is to be understood, however, that not all patients who undergo three consecutive injections will necessarily enter a stable period, e.g., not all patients respond well to the regimen of injections. However, if a patient does enter a stable period, the stable period lasts until the next big peak that requires medical attention. As an example, based on available scans for the patient of FIG. 2, one can identify three stable periods:
First stable period: 07/11/2017 to 11/3/2017 (This period follows the primary period of injection, after receiving first three consecutive injections);
Second stable period: 05/11/2018 to 08/15/2018 (This period follows three consecutive injections after a first large peak in February 2018);
Third stable period: 03/12/2019-03/17/2020 (This period follows three consecutive injections after a second large peak in September 2018).
   Considering the nature of retinal thickness changes during a dry period, and based on the inventor's data, the patient-personalized baseline thickness for each sector (macular ROI) may be defined as the average of the representative macular thickness measures of each corresponding sector during a dry period. It is to be understood that each patient has his/her own individualized, specific baseline based on personal eye physiology. If retinal thickness information during a patient's dry period is not available, then that patient's patient-personalized baseline thickness for each sector may be determined based on the average of the lowest representative macular thickness measures of each corresponding sector during treatment, known as the driest points.

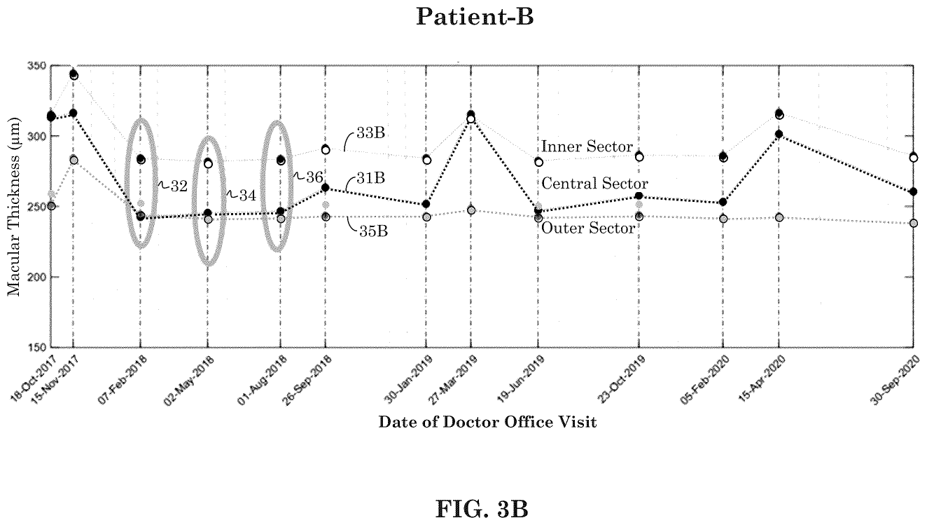

For example, FIGS. 3A and 3B show two additional examples from two respective patients (e.g., Patient-A and Patient-B), where reference characters 31A/B, 33A/B, and 35A/B identify respective plots of macular thickness corresponding to each of the three sectors 11, 13, and 15 identified in FIG. 1. Referring to FIG. 3A, the macular thickness plots of Patient-A show a patient history that includes a period of dry AMD, whose thickness data values are encircled by region 30. The respective averages of the data points of plot 31A, 33A, and 35A within region 30 may be used to define respective patient-personalized baseline (macular) thickness values for the Central Area, Inner Sector, and Outer Sector of Patient-A.

Referring to FIG. 3B, the macular thickness plots of Patient-B show a patient history that does not include a continuous dry AMD period prior to the onset of wet AMD. In other words, there is not enough history of Patient-B during a dry AMD period to define respective baseline macular thickness in a manner similar to that of FIG. 3A. In the case of FIG. 3B, a patient-personalized baseline (macular) thickness may be defined as the average thickness during “dry visits” or “driest visit” (highlighted by regions 32, 34, and 36). These visits may be doctor visits (e.g., check-up times) scheduled by a caregiver. In essence, FIG. 3B indicates that injections were administered almost from the first office visit, and thus there is no extended period (e.g., 3 consecutive office visits) of dry AMD. In this case, the lowest three corresponding data points of plots 31B, 33B, and 35B (e.g., the driest visits) are separately identified as data groups 32, 34, and 36, although injections were administered at these visits. Optionally, these may correspond to office visits when no injection was necessary, but is not limited to visits without injections. Further optionally, the identified regions may correspond to three consecutive doctor visits (e.g., OCT scans taken at predefined intervals corresponding to a previously scheduled macular thickness check-up times, e.g., a 4 to 6 week intervals), but again, it is not necessarily so limited and is not limited to consecutive visits. The respective averages of the data points of plot 31B, 33B, and 35B within regions 32, 34, and 36 may be used to define respective baseline macular thickness values for the Central Area, an Inner Sector, Outer Sector of Patient-B.

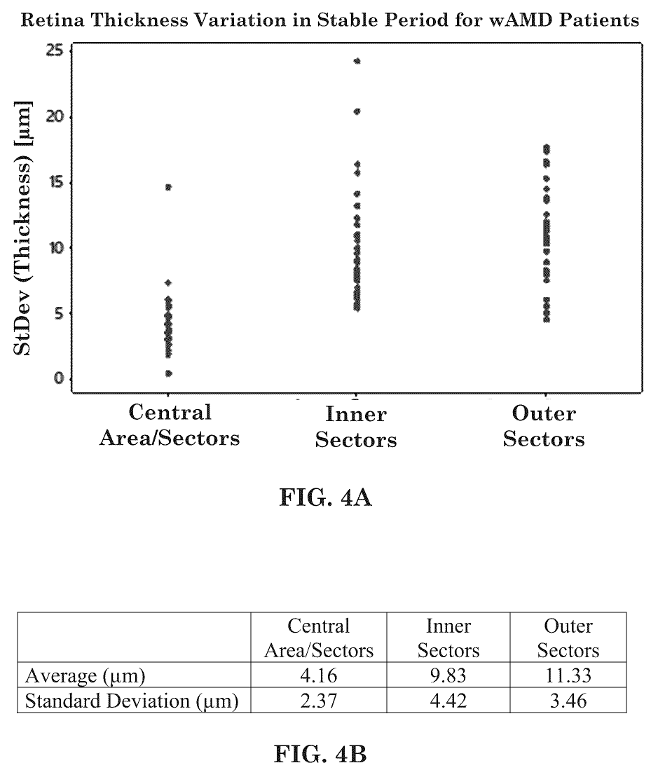

In addition to a patient-personalized baseline thickness, a normal/typical variation of retinal thickness (of a general population) for each of the three sectors (Central Area/Sector, Inner Sector, and Outer Sector) was also obtained. In an exemplary implementation, a normal variation in retinal thickness that does not require medical intervention was determined by reviewing retinal thickness change of each of the three sectors over 30 stable periods of the 22 wAMD patients. FIG. 4A illustrates the standard deviation of retinal thickness (variations) of representative retinal thickness measures for each of the three sectors during a stable period (e.g., periods 30, 32, 34 and 36 in FIGS. 3A and 3B) for the 22 wAMD patients. FIG. 4B shows a table that provides a summary of the normal retinal thickness variations over 30 stable periods of the 22 wet AMD patients. FIG. 4B provides the average macular thickness variation and standard deviation of thickness change for each of the Central Area/Sector, Inner Sector, and Outer Sector. A minimum threshold (e.g., offset) value may be defined from the table of FIG. 4B, e.g., may be determined based on the observed statistical deviation for each sector. This threshold (e.g., offset) value may then be used to define an upper specification limit (e.g., an upper threshold at which a thickness deviation may be deemed irregular (outside of the norm) and medication or medical attention may be recommended) and/or lower specification limit (e.g., a lower threshold that may be indictive of a dry period). For example, a patient's upper specification limit may be determined as the patient's patient-personalized baseline thickness plus a first offset (upper threshold offset), and the patient's lower specification limit may be determined as the patient's patient-personalized baseline thickness less a second offset (lower threshold offset). The first and second offsets maybe the same, or independently determined. For example, it has been observed that the Central Area/Sector is typically, but not necessarily, more sensitive to fluid buildup so although one would want to avoid false negatives (e.g., situations where the present system may recommend remote monitoring despite the patient having much fluid and in need of medical attention), one still may want to focus more on thickness variations in the Central Area/Sector. From FIG. 4A, it can be observed that a statistical deviation of 15 μm (e.g., the maximum/highest standard deviation in μm of the Central Area/Sector) may be sufficient to accommodates normal thickness variations not only in the Central Area/Sector, but also avoid most false negatives in the Inner Sector and Outer Sector. In this case, a select one sector (the Central Area/Sector) may be used to set a common offset to determine the upper specification limit (and/or the lower specification limit) for all sectors (macular ROIs). Alternatively, another statistical deviation, such as or 6 to 7 standard deviations, as shown in FIG. 4B, may be used for any, or all, sectors.

The above analysis is beneficial for determining/identifying normal variations in a patient's retinal thickness that may not require (e.g., immediate) medical intervention (e.g., injection of anti-VEGF medication or a medical office visit). That is, the present system may monitor a patient's medical condition. Using this analysis, the present system may set a (e.g., patient-optimized or individualized) threshold (i.e., an upper specification limit) to identify a change in retinal thickness that may indicate that a patient may benefit from (e.g., it is advisable that the patient) visit a doctor's office for further medical analysis, at which point, a health care provider may determine if the patient's condition has significantly changed and might benefit from medical treatment (e.g., injection of anti-VEGF medication). For example, the present system may inform the patient that a doctor's visit is advisable, or may use a communication network to issue an automatic alert to a doctor (or predefined medical provider) or send a request for a doctor's appointment. The communication network may be a wired or wireless communication network, such as the Internet, a cellular network, etc. Applicants have determined that an individualized (patient-specific) baseline and threshold for each patient is contingent on the patient's disease status, fluid type, and response of the patient's eye to treatment. For example, some patients may inherently have thicker retinal tissue, and thus require a higher baseline than patients with inherently thinner retinal tissue. The present system, in addition to taking the above-mentioned parameters into account when setting a patient's initial upper specification limit (macular thickness threshold), further adjusts (e.g., changes/updates) the threshold parameter during the course of treatment (e.g., based on a patient's personal response to treatment), and further permits this parameter to be manually adjusted by retina specialists, doctor, or other authorized medical caregiver. For example, the present system may suggest a personalized threshold for a specific patient, but the patient's doctor may override the system-suggested (e.g., patient-specific upper and/or lower) threshold and adjust the threshold manually. In some embodiments, the threshold may be adjustable (e.g., by a doctor, technician, or other authorized user) in intervals of fixed size within a recommended range, such as a threshold range of 15 to 30 μm in incremental values of 15, 20, 25, or 30 μm. However, higher or lower threshold values outside of the system's suggested range (and not limited to the system-recommended incremental values) can also be set by the doctor based on the doctor's opinion.

By implementing the analysis in this work in a remote monitoring OCT system, the present system can personalize treatment on a patient-by-patient basis, which is well-suited for personal, remote, or home OCT applications. Moreover, based on the results of the present study, the present system is able to identify excessive amounts of fluid (e.g., retinal fluid leakage or retinal fluid build-up) early and induce/recommend treatment in a timely manner. Additionally, the present system's ability to review a patient's OCT database/history (e.g., previous scan results may be stored within the remote OCT system) permits the system to analyze a patient's individualized response to treatment and predict/recommend better selection of drugs and treatment intervals for that patient.

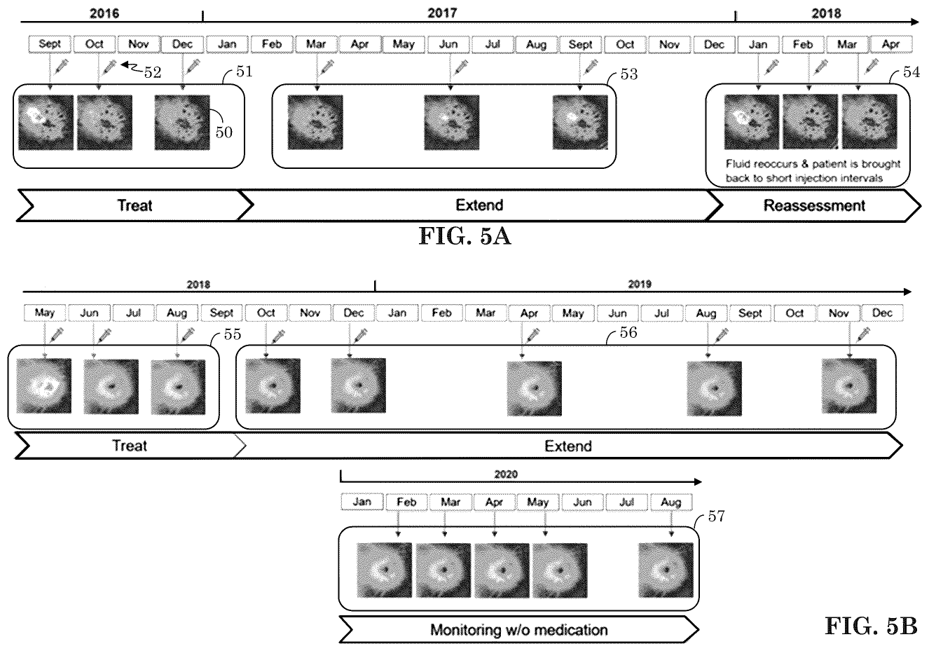

To better illustrate some of the above-mentioned applications, FIGS. 5A and 5B provide an overview of injections and office visits of two other wAMD patients over a two-year period (FIG. 5A) and a three-years (FIG. 5B). These two examples illustrate how remote monitoring of wet AMD could help provide more information to physicians. FIG. 5A is a showcase of doctor visits by the first wAMD patient over the two-year period with examples of thickness maps 50 taken at each doctor visit. In 2016, the first wAMD patient began regular office visits, and initially received frequent injections, as indicated by period 51 and injection symbols 52. In 2017, due to good response to medication, the first wAMD patient's physician applied a treat-and-extend protocol, wherein the time spans between injections were increased to an as-needed basis, as indicated by period 53. However, after some time, there was a massive fluid leakage in the first wAMD patient's retina that was not identified until January 2018, see period 54. Due to a lack of data between September 2017 to January 2018, this massive leakage was not recognized until it had reached a later/advanced stage. If remote monitoring of wet AMD had been available to the first wAMD patient, the massive leakage may have been captured/identified earlier and the prospects for preventing permanent vision loss would have been improved.

FIG. 5B provides an example of doctor office visits by the second wAMD patient whose overview period spans three-year. Like in the example of FIG. 5A, the second wAMD patient also initially received frequent injections during an initial period 55, and then transitioned to a treat-and-extend protocol period 56, wherein the time between injections were increased to an as-needed basis. However, due to a good response to treatment, the second wAMD patient did not require/receive (injection) medication in 2020. Nonetheless, the second wAMD patient still had to be brought regularly to the doctor's office for monitoring, as indicated by period 57. Patients with wet AMD are often elderly, to whom being brought to a doctor's office may be a difficulty and a burden. If remote monitoring of wAMD were available to the second wAMD patient, the burden of frequent and regular office visits could have been eliminated, or mitigated.

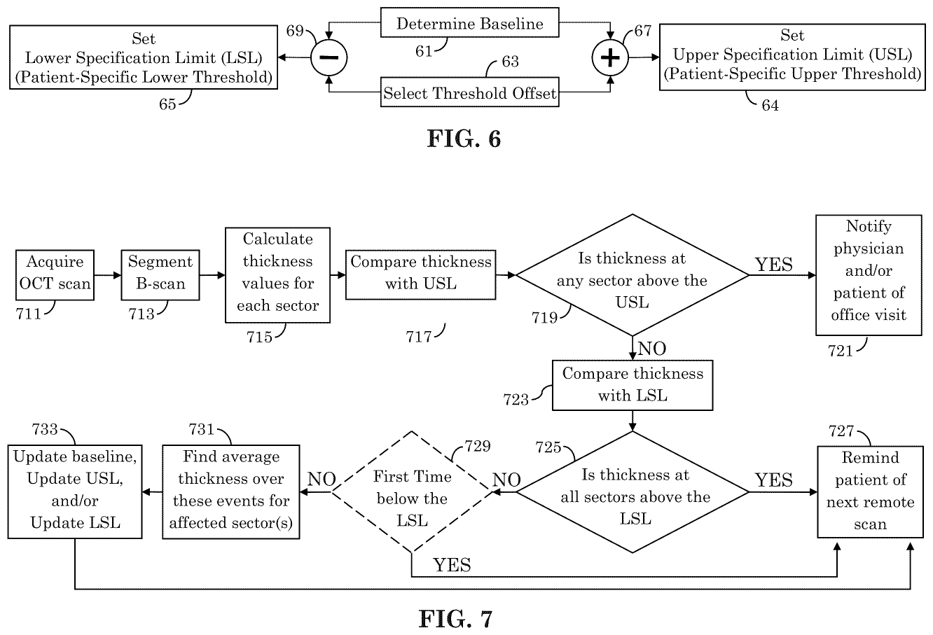

FIGS. 6 and 7 illustrate two stages of an exemplary workflow for remote monitoring of fluid leakage in wAMD patients in accord with the present invention. FIG. 6 shows an initialization stage in which a remote monitoring system in accord with the present invention is initialized/prepared for home/remote use. In order to implement remote monitoring of retinal fluid leakage of a wet AMD patient, a first step 61 is to determine a personalized baseline of retinal thickness (e.g., patient-personalized baseline thickness) for one or more, and preferably, of the three above-described eye sectors (see FIG. 1) for the specific patient. This step can be done based on the medical history of doctor office visits by a patient during a dry AMD period, if available, or based on doctor visits with minimum amount of fluid. This baseline can be adjusted as doctor-visit data accumulates. Alternatively, since this data is available to the physician, the physician (or designated technician) can determine a personalized baseline for the patient. Further alternatively, the physician may choose from a list of recommended threshold values provided by the present system. A next step 63 is to assign a threshold offset for each eye sector (e.g., each of the Central, Inner, and Outer sectors). The threshold offset can be the same for all sectors (for e.g. 15 μm) or can be varied among the sectors (for example, 15 μm for the central sector, 25 μm for inner the sector, and 20 μm for the outer sector). The upper specification limit (USL) 64 and lower specification limit (LSL) 65 for each eye sector may be determined based on the determined baseline value(s) and selected threshold offset(s). For example, the upper specification limit 64 for each eye sector may be set by adding each sector's corresponding patient-personalized baseline thickness value and its selected (e.g., upper) threshold offset (step 67), and the lower specification limit 65 for each eye sector may be set by subtracting each sector's corresponding selected (e.g., lower) threshold offset from its corresponding patient-personalized baseline thickness value (step 69).

After the initial setup sequence of FIG. 6, the active remote monitoring method of FIG. 7 can begin. In step 711, a patient may use a personalized/portable OCT system in accord with the present invention to remotely acquire/collect an OCT scan of the patient's eye. The scan data is then (automatically by the portable OCT system) segmented, e.g., by retinal layer(s), (step 713), and retinal thickness values (e.g. a thickness map is created) of at least one, and preferably all, of the three designated eye sectors (e.g., the central, inner, outer sectors) are calculated (step 715), and a respective representative macular thickness value of each sector (e.g., based on individual thickness measures within a sector or a sector-average thickness measure) gets compared to its respectively designated upper specification limit (USL) (step 717). Techniques for segmenting OCT data and determining thickness values from the segmented data are well known and need not be discussed here. If the representative macular thickness value/measure of any of designated eye sector is above its respective upper specification limit (step 719=YES), the device issues a flag/signal and notifies the physician (and/or patient), locally or remotely, of the potential need for a doctor office visit (step 721). If none of the sectors have a representative macular thickness measure above their respective upper thresholds (step 719=NO), then representative macular thickness measures get compared to their respective lower specification limit (LSL) (step 723). If all thickness measures are above their respective lower specification limit (step 725=YES), then the present scan sequence is completed, a report can be generated, and the patient may be issued a notification of the next scheduled remote scan (step 727). If the representative macular thickness measure of any eye sector is not above its respective lower specification limit (step 725=NO), the present system may optionally check if this is the first time that any thickness measure is below its respective lower specification limit (step 729), if it is (Step 729=YES), the process again proceeds to step 727 and the present scan sequence is completed, a report can be generated, and the patient may be issued a notification/reminder of the next scheduled remote scan. If this is not the first time that a thickness measure is below its respective lower specification limit (Step 729=NO), then the system can check the number of consecutive times (e.g., consecutive, scheduled OCT scans) that the representative macular thickness measures have been below their respective lower specification limits, and update the patient's patient-personalized baseline thickness, upper specification limit (USL), and/or lower specification limit (USL) (see FIG. 6) for any of the affected sectors (e.g. eye sector(s) whose representative macular thickness value(s)/measure(s) have been below their respective specification limit). For example, if the thickness measure in any of the three eye sectors is below its respective lower specification limit for two or more (e.g., at least three) consecutive sessions, this may be an indication that the patient's eye is responding well to treatment and the patient-personalized baseline thickness of the affected sector(s) may be changed to the average thickness of scans with low values of the affected sector(s) (e.g., the average of the thickness measure(s) below their respective lower specification limit in the three consecutive scans of the affected sector, or the average of all the thickness measures within the affected sector(s)), step 731. At this point, the patient-personalized baseline thickness may be updated, and optionally also the upper and/or lower specification limits can be changed (step 733), either automatically or depending upon doctor's approval/input. If automatically, the upper and/or lower specification limit may be changed to the next higher or lower incremental value within the recommended range, or by incorporating the patient's thickness measures with low values into the statistical analysis for determining the upper and/or lower threshold offsets, as discussed above. Alternatively, the present remote OCT system may optionally inform the doctor remotely (e.g., via the Internet or other telecommunication system or network) of the patient's reduced thickness measures and its recommendation(s) for adjusting the baseline, upper specification limit, and/or lower specification limit. The doctor may then remotely instruct the present OCT system to update its baseline, upper specification limit, and/or lower specification limit. The remote OCT system then proceeds to step 727 to end the present session.

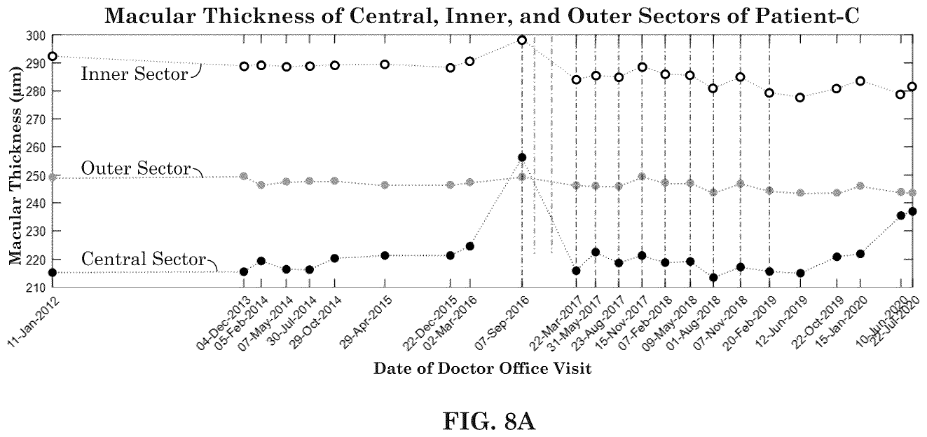

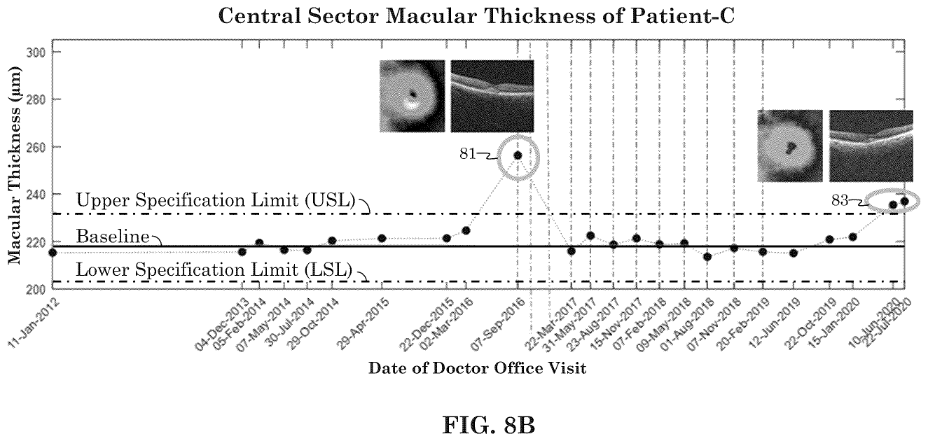

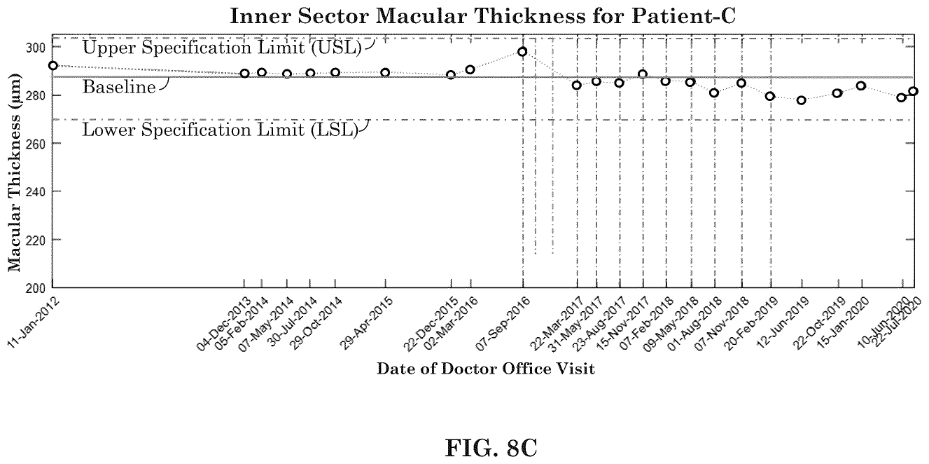

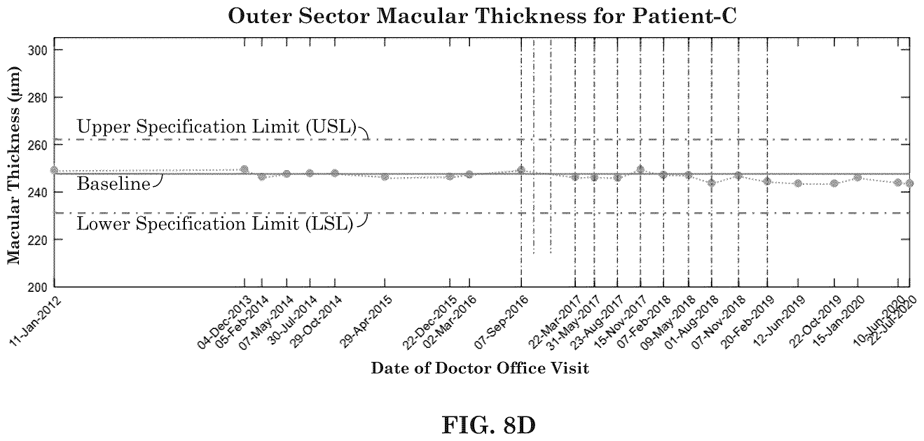

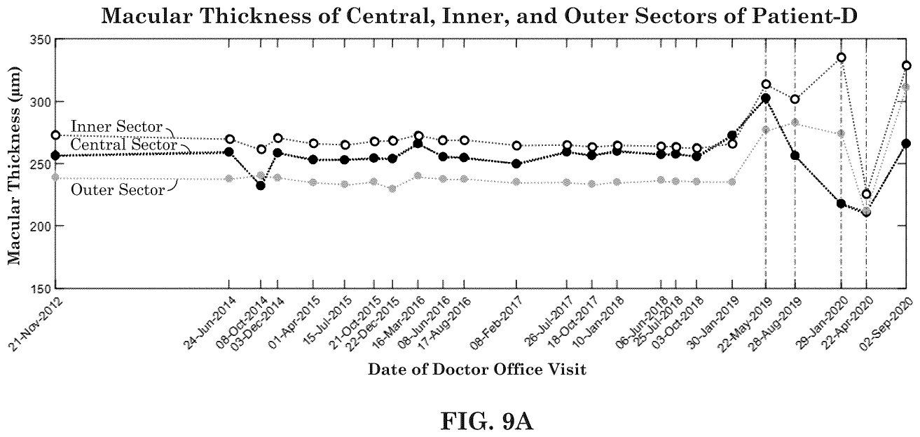

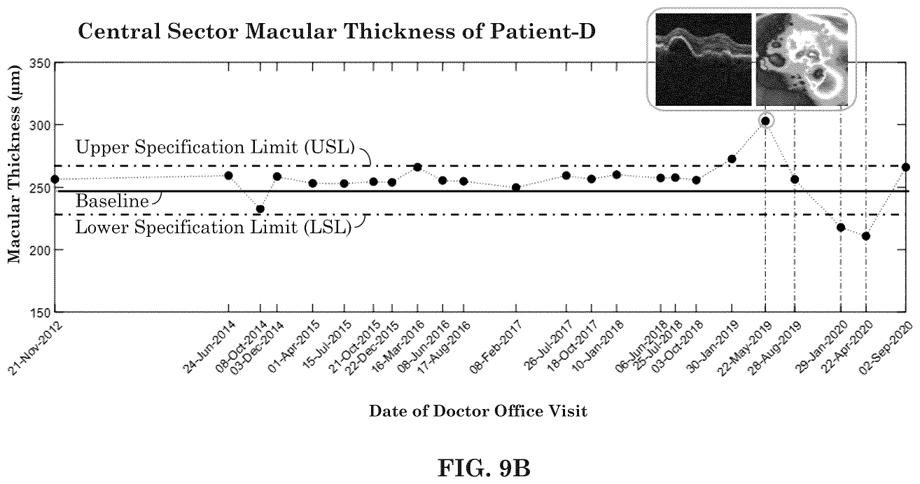

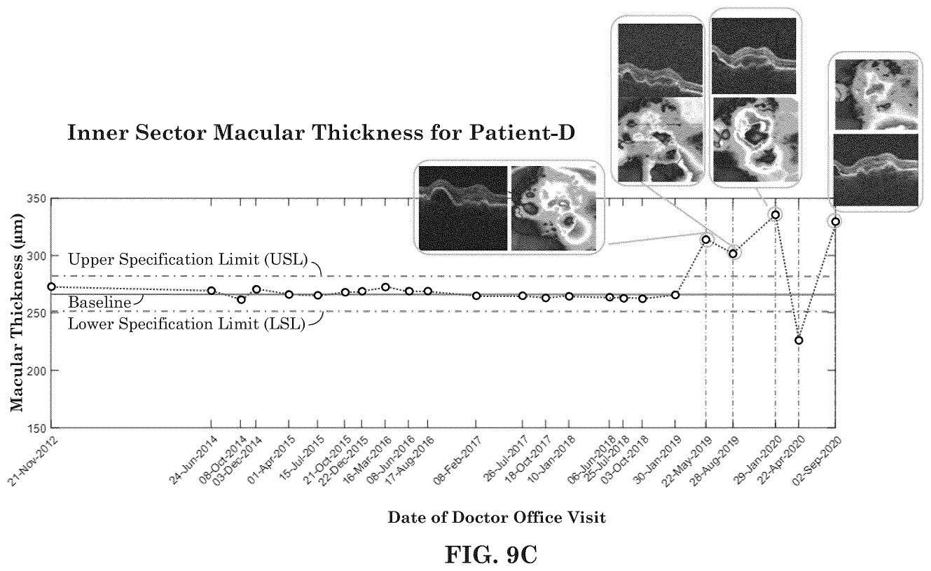

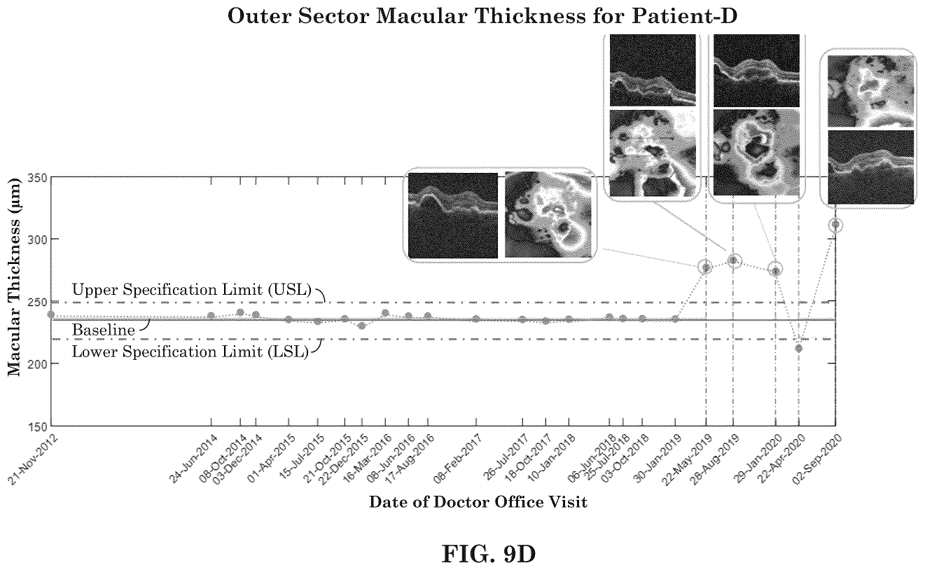

FIGS. 8A-8D and 9A-9D illustrate how application of the present invention to two exemplary test patients (Patient-C and Patient-D, respectively) could have identified when there was an absolute need for medical intervention, and when it would not have been necessary for the patients to visit their doctor's office. The present example applies the automatic procedures of the present invention without any doctor's input since it is being applied retroactively to existing medical history as an example of its effectiveness. FIGS. 8A to 8D show the retinal thickness levels of wAMD Patient-C over an eight-year period. FIG. 8A shows the representative macular thickness measure determined based on an OCT scan (e.g., taken at a doctor's office visit, as noted by date) for each of the three monitored retinal sectors; the central sector, the inner sector, and the outer sector. This data was acquired during Patient-C's office visits, with the dates of OCT scans listed and the times when injections (e.g., of anti-VEGF medication) were administered noted by vertical, dotted lines. Patient-C was initially diagnosed with dry AMD for a period that spanned the first four years, from 11-Jan-2012 to 2 Mar. 2016. Six months later on 7 Sep. 2016, Patient-C received a first injection, as noted by the vertical dotted line. Following this initial injection, Patient-C received multiple injections in about three-month intervals for the next two and a half years, receiving the last injection during this observation period on 20 Feb. 2019. Although Patient-C was then off the medication, Patient-C still had to continue office visits for the remainder of the present observation period to receive OCT scans and monitor the progress of any fluid leakage.

Applying the present invention to the medical history of Patient-C, for each of the central, inner, and outer sectors, a patient-personalized baseline was defined as the average of retinal thickness during the stable period (e.g., during Patient-C's dry AMD period). The present example used an upper and lower threshold offset of 15 microns from the respective patient-personalized baseline thickness of each of the central, inner, and outer sectors to determine its respective upper specification limit and lower specification limit. For ease of illustration, FIG. 8B superimposes the (patient-personalized) baseline (thickness), patient-specific upper threshold (upper specification limit), and patient-specific lower threshold (lower specification limit) of the central sector (as determined using the present invention, see for example, FIGS. 6 and 7) over the macular thickness data of the central sector from FIG. 8A. Similarly, FIG. 8C shows the baseline (thickness), patient-specific upper threshold (USL), and patient-specific lower threshold (LSL) over the inner sector macular thickness data, and FIG. 8D shows the determined baseline (thickness) and patient-specific upper and lower thresholds over the outer sector macular thickness data.

In the present example, the macular thickness of Patient-C's inner and outer sectors remained within their respective upper and lower thresholds, as shown in FIGS. 8C and 8D. However, Patient-C experience significant leakage primarily in the central sector. Using the present threshold of 15 microns, FIG. 8B identifies three instances (circles 81 and 83) wherein macular thickness was above the upper threshold in the central sector. For illustration purpose, macular thickness values, B-scans, and thickness maps for two of these instances are shown. These events would be triggers indicating the necessity for an injection, or at least for direct medical intervention/examination.

In the case of Patient-D, the second exemplary test patient, FIGS. 9A to 9D show the retinal thickness levels over an eight-year period. FIG. 9A shows representative macular thickness measurements determined by OCT scans for each of the three monitored retinal sectors; the central sector, the inner sector, and the outer sector. In a manner similar to that of FIG. 8A, this data was acquired during Patient-D's doctor office visits, with the dates of OCT scans listed and the occasions when injections were administered noted by vertical, dotted lines. Referring to FIG. 9A, Patient-D is shown to have had dry AMD for the first seven years from 21 Nov. 2012 to about 30 Jan. 2019. However, Patient-D did not receive the first injection until about four months later on 22 May 2019. Patient-D continued injection treatment for the remainder of the present eight-year observation period.

Like in the above example, FIGS. 9B, 9C, and 9D respectively show Patient-D's central sector, inner sector, and outer sector with their determined baseline, upper specification limit, and lower specification limit. Each respective (patient-personalized) baseline is defined as the average of (representative) retinal thickness measures determined at doctor visits with dry AMD status (optionally, a running average of 3 consecutive visits). As in the first example, an offset of 15 μm from the baseline is set to define the upper and lower specification limit of each eye sector.

A majority of Patient-D's fluid leakage is in a peripheral area, but the present 15 μm threshold for all sectors identifies the first triggering event in the central sector on 30-Jan-2019, as shown in FIG. 9B. Multiple other triggering events based on macular thickness are subsequently also found in the inner and outer sectors, as shown in FIGS. 9C and 9D, respectively. Had the present system been in use during the observation period of Patient-D, the early triggering event on 30 Jan. 2019 would have been flagged as significant and requiring an injection of medication. Patient-D might then have started medication treatment four months earlier than he/she did. As it known in the art, early diagnosis and treatment of wet AMD are crucial since vision loss due to fluid leakage is not currently recoverable.

Thus, the present invention provides several benefits. The present system/method provide for monitoring of retina stability and fluid reoccurrence events based on statistical analysis of retinal thickness measurements, such as obtained by OCT scans and/or thickness maps. The present monitoring system is able to raise a flag when there is significant amount of fluid in patient's retina based on clinician set thresholds, or on automatically determined/calculated threshold or on pre-set fixed-value thresholds. Additionally, the present monitoring system provides for a baseline and threshold (indicative of when an injection is needed) that can be individually set on a per patient basis, and can be further updated as a patient accumulates additional macular thickness measurements in subsequent OCT scans. Thus, the present monitoring system can adjust the baseline and thresholds when a patient's eye is responding well to treatment, as determined by macular thickness changes. The present monitoring system can also help physicians to understand a patient's individualized response to treatment and induce better selection of drugs and treatment intervals for the patient. For example, in the present monitoring system, optimization of patient-treatment schedule may be determined based on statistical variations of retinal thickness change.

Hereinafter is provided a description of various hardware and architectures suitable for the present invention.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

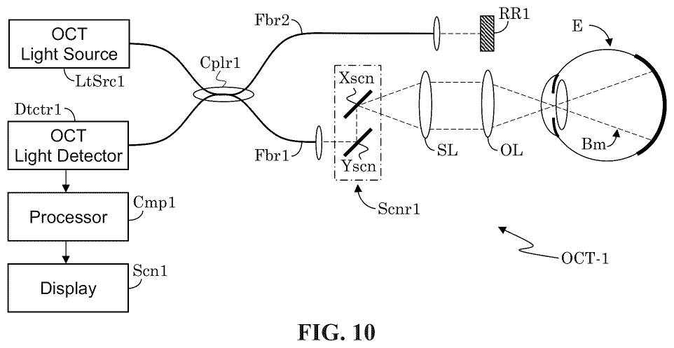

FIG. 10 illustrates a generalized frequency domain optical coherence tomography (FD-OCT) system used to collect 3D image data of the eye suitable for use with the present invention. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrcl may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produce substantially collimated light, and ophthalmic lens OL may then focus onto the sample. The present example illustrates a scan beam that needs to be scanned in two lateral directions (e.g., in x and y directions on a Cartesian plane) to scan a desired field of view (FOV). An example of this would be a point-field OCT, which uses a point-field beam to scan across a sample. Consequently, scanner Senr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner may be needed to scan the line-field beam across the sample to span the desired FOV. If the scan beam were a full-field beam (e.g., a full-field OCT), no scanner may be needed, and the full-field light beam may be applied across the entire, desired FOV at once.

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RRI with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 14. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy-Holographic Optical Coherence Tomography,” Optics Letters, 36 (13): 2390 2011; Y. Nakamura, et al, “High-Speed Three Dimensional Human Retinal Imaging by Line Field Spectral Domain Optical Coherence Tomography,” Optics Express, 15 (12): 7103 2007; Blazkiewicz et al, “Signal-To-Noise Ratio Study of Full-Field Fourier-Domain Optical Coherence Tomography,” Applied Optics, 44 (36): 7722 (2005)). In time-domain systems, the reference arm needs to have a tunable optical delay to generate interference. Balanced detection systems are typically used in TD-OCT and SS-OCT systems, while spectrometers are used at the detection port for SD-OCT systems. The invention described herein could be applied to any type of OCT system. Various aspects of the invention could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj (k)). The real-valued spectral data typically goes through several post-processing steps including background subtraction, dispersion correction, etc. The Fourier transform of the processed interferogram, results in a complex valued OCT signal output Aj (z)=|Aj|eiφ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

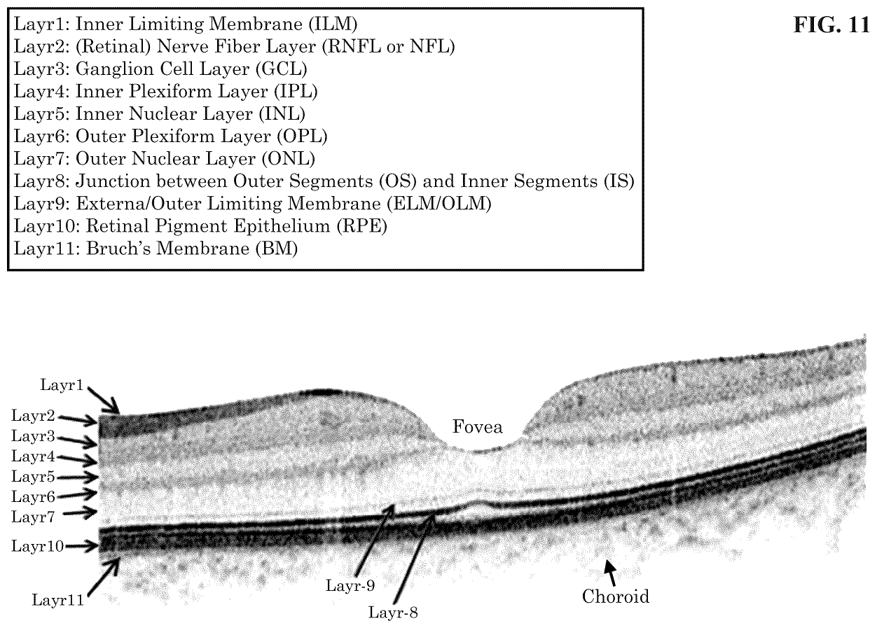

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 11. An OCT B-scan of the retinal provides a view of the structure of retinal tissue. For illustration purposes, FIG. 11 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

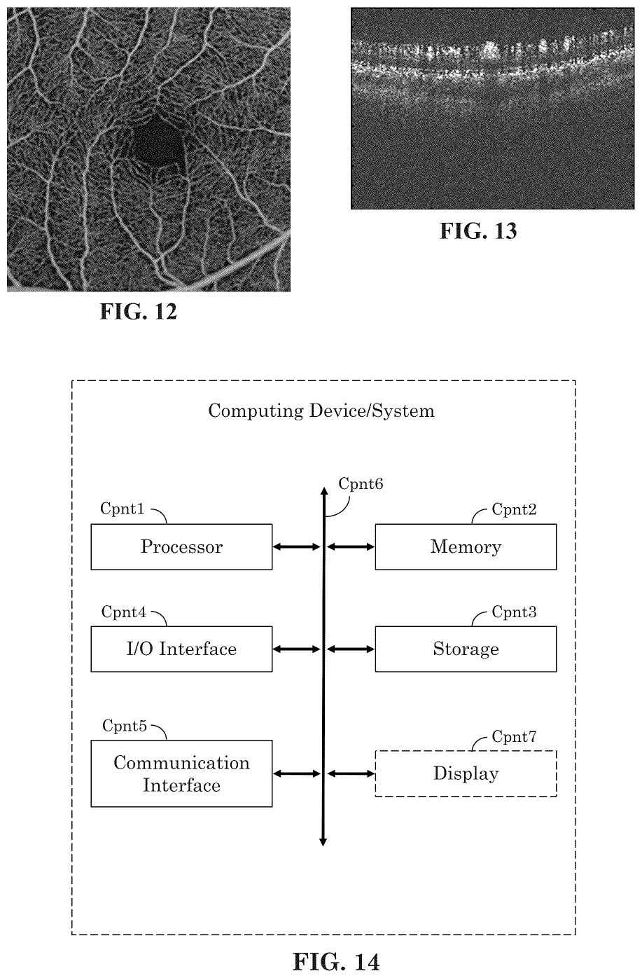

FIG. 12 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 13 shows an exemplary B-scan of a vasculature (OCTA) image. As illustrated, structural information may not be well-defined since blood flow may traverse multiple retinal layers making them less defined than in a structural OCT B-scan, as shown in FIG. 11. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique requiring the injection of a dye to observe vascular flow in the retina. Additionally, in dry age-related macular degeneration, OCTA has been used to monitor a general decrease in choriocapillaris flow. Similarly, in wet age-related macular degeneration, OCTA can provide a qualitative and quantitative analysis of choroidal neovascular membranes. OCTA has also been used to study vascular occlusions, e.g., evaluation of nonperfused areas and the integrity of superficial and deep plexus.

### Computing Device/System

FIG. 14 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the invention has been described in conjunction with several specific embodiments, it is evident to those skilled in the art that many further alternatives, modifications, and variations will be apparent in light of the foregoing description. Thus, the invention described herein is intended to embrace all such alternatives, modifications, applications, and variations as may fall within the spirit and scope of the appended claims.

1. A method of monitoring a medical condition of an eye of a patient, comprising:
collecting, by an optical coherence tomography (OCT) system, an OCT scan of the eye;
designating, by a processor, a macular region of interest (ROI) within the OCT scan;
determining, by the processor, a representative macular thickness measure that is characteristic of the overall thickness of the macular ROI;
determining, by the processor, a patient-personalized baseline thickness for the macular ROI;
defining, by the processor, an upper specification limit based on the patient-personalized baseline thickness;
defining, by the processor, a lower specification limit based on the patient-personalized baseline thickness;
in response to the representative macular thickness measure being higher than or equal to the upper specification limit, issuing, by the processor, an electronic signal indicating a need for medical attention; and
in response to the representative macular thickness measure being lower than or equal to the lower specification limit, adjusting, by the processor, at least one of the patient-personalized baseline thickness, the upper specification limit, or the lower specification limit based on the representative macular thickness measure.

2. The method of claim 1, wherein the representative macular thickness measure is based on a plurality of individual macular thickness measures within the macular ROI, and the representative macular thickness measure is determined as the average of all the individual macular thickness measures within the macular ROI, the highest individual macular thickness measure within the ROI, or the average of two or more of the highest individual macular thickness measures within the ROI.

3. The method of claim 1, wherein the patient-personalized baseline thickness is based on an average of a predefined number of lowest, previous representative macular thickness measures of the macular ROI determined according to caregiver-scheduled, macular thickness check-up times.

4. The method of claim 3, wherein the lowest, previous representative macular thickness measures are extracted only from stable periods, wherein the medical condition is age-related macular degeneration (AMD) in the eye and a stable period is defined as one or more of a dry AMD period, which is a period where individual macular thickness measures of the macular ROI did not vary by more than 5%, or a period wherein no injection of medication was administered to the eye.

5. The method of claim 1, wherein at least one of the upper specification limit or the lower specification limit is defined as an offset from the patient-personalized baseline thickness, and the offset is based on a statistical analysis of a population of macular thickness measures from corresponding macular ROIs in a general population of test eyes taken during stable periods, wherein the medical condition is age-related macular degeneration (AMD) in in the eye and a stable period is defined as a dry AMD period, which is a period wherein a test eye's individual macular thickness measures do not vary by more than 5%, or a period wherein no injection of medication was administered to a test eye.

6. The method of claim 5, wherein the statistical analysis provides a statistical deviation among the population of macular thickness measures, and the offset is based on a maximum statistical deviation.

7. The method of claim 1, wherein the patient-personalized baseline thickness is user adjustable in intervals of fixed size within a predefined range, the user adjusted patient-personalized baseline overriding any previous determined of the patient-personalized baseline thickness.

8. The method of claim 1, wherein in response to the representative macular thickness measure being lower than or equal to the lower specification limit, the patient-personalized baseline thickness is adjusted to an average of the current representative macular thickness measure and one or more previous macular thickness measures of the eye that are also lower than or equal the lower specification limit.

9. The method of claim 1, wherein the patient-personalized baseline thickness is adjusted if the current representative macular thickness measure that is lower than or equal to the lower specification limit is the third, or more, consecutive representative macular thickness measure of the eye that is lower than or equal to the lower specification limit.

10. The method of claim 1, wherein the at least one of the upper specification limit or the lower specification limit is adjusted independent of the patient-personalized baseline thickness.

11. The method of claim 1, wherein the at least one of the patient-personalized baseline thickness, the upper specification limit, or the lower specification limit is automatically adjusted by a preset incremental amount within a predefined range.

12. The method of claim 1, wherein the at least one of the upper specification limit or the lower specification limit, is automatically adjusted by incorporating the value of the representative macular thickness measure into a recalculation of the at least one of the upper specification limit or the lower specification limit.

13. The method of claim 1, wherein a plurality of the macular ROIs are designated, each having a respective of the patient-personalized baseline thickness independent of each other.

14. The method of claim 13, wherein the plurality of the macular ROIs have different respective upper specification limits and lower specification limits.

15. The method of claim 13, wherein the upper offset and lower offset of a select one of the plurality of the macular ROIs is set as the upper offset and lower offset of all of the plurality of macular ROIs.

16. The method of claim 13, wherein the plurality of the macular ROIs are concentric to each other.

17. The method of claim 16, wherein:
each of the plurality of the macular ROIs has a respective of the upper specification limit and the lower specification limit based on a corresponding upper offset and lower offset from its respective patient-personalized baseline thickness; and
the upper offset and lower offset of the center-most ROI is set as the upper offset and lower offset of all of the plurality of macular ROIs.

18. The method of claim 1, wherein the at least one of the upper specification limit, the lower specification limit, or the patient-personalized baseline thickness is remotely adjustable by an authorized user in response to receiving an electronic message over a telecommunication network.

19. The method of claim 1, wherein the OCT scan of the eye is collected using a self-applied optical coherence tomography system.

20. An optical coherence tomography (OCT) system comprising:
a light source for generating a beam of light;
a beam splitter having a beam-splitting surface for directing a first portion of the light into a reference arm and a second portion of the light into a sample arm;
optics for directing the light in the sample arm to one or more locations on a sample;
a detector for receiving light returning from the sample and reference arms and generating signals in response thereto; and
the OCT system being characterized by a processor configured to implement the method of claim 1.
